(19) United States
(12) Patent Application Publication
(10) Pub. No.: US 2024/0374143 A1
(43) Pub. Date: Nov. 14, 2024

(54) METHOD AND DEVICE FOR MEASURING BILIRUBIN

(71) Applicant: Jan Oliver Haarer, Winterthur (CH)

(72) Inventors: Jan Oliver HAARER, Winterthur (CH); Christiane HAARER, Winterthur (CH)

(73) Assignee: Jan Oliver Haarer, Winterthur (CH)

(21) Appl. No.: 18/692,370

(22) PCT Filed: Sep. 15, 2021
(86) PCT No.: PCT/EP2021/025348

§ 371 Date: May 15, 2024

(51) Int. Cl. A61B 5/00; A61B 5/145; A61B 5/1455

(52) U.S. Cl. CPC A61B 5/0071; A61B 5/0075; A61B 5/14546; A61B 5/1455; A61B 5/7282

(57) ABSTRACT

A method for determining bilirubin, in which light of a wavelength suitable for the transformation of bilirubin is irradiated into a living tissue region, light from the irradiated living tissue region is detected, and a time sequence of detection signals changing according to the transformation is determined, and bilirubin is determined in response to the time sequence, wherein light is locally irradiated into the living tissue region, the intensity of which is high enough to locally degrade a more rapidly phototransformable form of bilirubin, in comparison with a more slowly phototransformable form of bilirubin, regardless of the body's own transport processes, relative to other tissue regions, and different forms of bilirubin in the non-degraded state are quantified from the detection signals of the time sequence detected during the degradation. For the improved total bilirubin measurement, multispectral measurements are also proposed.

### FIELD OF THE INVENTION

The present invention relates to a method and to a device for the transcutaneous measurement of bilirubin.

### BACKGROUND

Bilirubin is present in the liver and spleen when the heme portion of hemoglobin is degraded, and occurs in the blood serum and in tissue in all humans. If bilirubin values are significantly increased, this is critical because high bilirubin concentrations are toxic. For the layman, an excessively high bilirubin concentration can be seen, for example, as jaundice, with a known, yellowish skin tone, as often occurs in newborns, and also in adults.

Bilirubin can be present in various forms in the human body. For example, bilirubin is modified in the liver and converted into its water-soluble form bilirubin diglucuronide. Since this form of bilirubin can be excreted directly, it is also referred to as direct bilirubin, in contrast to so-called indirect bilirubin, whose designation does not relate to a single molecule, but rather to non-metabolized forms of bilirubin, the most important entity of which is the Z,Z isomer, which is not water-soluble and can therefore not be excreted directly from the body. This is why the designation indirect is used.

In the human body, the majority of bilirubin is non-covalently bound to a protein (albumin) (so-called indirect bilirubin or indirectly reacting bilirubin). In addition, there is water-soluble, i.e., glucuronidated (di- and mono-glucuronidated) bilirubin, and delta bilirubin (so-called direct bilirubin, directly reacting bilirubin or free bilirubin) fixedly (covalently) bound to albumin. Accordingly, a distinction can be made between free bilirubin, bilirubin bound to albumin, and bilirubin conjugated in the liver. However, in a generally simplified manner, reference is made only to a single bilirubin concentration.

Since the critically increased bilirubin concentrations usually occur due to functional disorders, bilirubin is an important diagnostic marker which is used, for example, in clinical trials in order to be able to estimate the liver toxicity of drugs, in particular in the development of new drugs. However, it also plays a great role in the diagnosis of various diseases, such as newborn jaundice.

The presence of an increased bilirubin concentration is therefore an important diagnostic indicator. Further important diagnostic indications can be obtained if it is known which of the forms of bilirubin are present to an increased degree; for instance, increased direct (glucuronidated) bilirubin is indicative of a bile duct problem or a problem in the intestine. With this type of problem, the liver is capable of glucuronidation, but the direct bilirubin cannot be moved into the intestines. Conversely, increased values of indirect bilirubin can indicate liver damage.

By way of example, as far as the different forms of bilirubin are concerned, it may be noted that an increase in unconjugated bilirubin can occur due to hemolysis. Further tests can indicate an Rh, ABO, or Kell incompatibility, and/or an immunohemolysis can be distinguished from innate hemolytic anaemias. Furthermore, an increase in unconjugated bilirubin can also be attributed to larger hematomas, a disruption in the uptake of bilirubin into the liver cells due to a physiological newborn jaundice or a polycythemia, hormones, hypothyroidism, drug administration, or Gilbert-Meuengracht's syndrome, and for example to a disorder of the glucuronidation in the immature liver of a premature infant, which is referred to as Crigler-Najjar syndrome.

An increase in conjugated (directly reacting) bilirubin, in contrast, is attributed to diseases of the liver, such as hepatitis, autoimmune hepatitis, metabolic defect or toxic liver damage, certain drugs and parenteral nutrition, or diseases of the bile ducts, such as intrahepatic biliary canal dis/hypoplasia, extrahepatic biliary duct atresia, gall bladder stones, gall bladder cysts, cholangitis, or so-called rotor syndrome or Dubin-Johnson syndrome.

This demonstrates that a distinction between the different forms of bilirubin can offer considerable advantages.

Due to the toxicity of bilirubin, increased bilirubin concentrations must also be counteracted. This occurs, especially in newborns, by irradiating the skin with blue light. When the skin is irradiated with light at the absorption maximum of the bilirubin, which is at 460 nm, without glucuronidation, this form of bilirubin can be degraded via various pathways. The most important pathway is structural isomerization, which leads to the formation of lumirubin. Configurational photoisomerization, in which an untoxic water-soluble bilirubin molecule arises from the toxic hydrophobic bilirubin, is less important. Photooxidation can also be brought about, wherein dipyrroles arise. However, this plays a quantitatively less significant role in phototherapy for newborns with blue light.

Against this background, it would be desirable to be able to quickly measure bilirubin in a simple manner. Non-invasive measuring methods that allow rapid and precise measurement would be particularly desirable. Furthermore, it would be desirable to have at least one indication of which of the forms of bilirubin are elevated, in each case.

Methods and devices for measuring bilirubin are already known. It has been proposed to carry out measurements optically by irradiation of light into the body and detecting the light that then can be received from the body.

According to DE 10 2016 014 071 A1, bilirubin for therapies can be measured transcutaneously by irradiating light onto a living tissue portion as part of an exposure step, and at least a portion of the light exiting from this tissue portion is detected, and then intensity and wavelength are taken into account in a system of equations with which the concentration of bilirubin is determined. Concentrations of hemoglobin, and the skin tissue, are taken into account by virtue of the fact that absorption values of hemoglobin are determined at wavelengths of 452 nm and 500 nm because these are the isosbestic wavelengths at which the absorption by hemoglobin is not altered by its oxygenation/de-oxygenation.

DE 10 2017 008 631 A1 discloses a therapy method in which a sensor device connected to a living tissue region of the patient comprises a light source and a detection device with which light exiting the living tissue region of the patient is detected. Signals are generated via the detection device which enable a conclusion to be drawn about the bilirubin concentration in the living tissue region, which makes it possible to operate the arrangement taking into account the determined bilirubin concentration, and to observe a gradual decrease in the bilirubin concentration.

The paper Neonatal wearable device for colorimetry-based real-time detection of jaundice with simultaneous sensing of vitals by Go Inamori et al. in Sci. Adv. 2021, 7: eabe3793, Mar. 3, 2021, discloses the determination of bilirubin concentrations by determining the difference in the absorption of green and blue light that is generated with small LEDs. The paper demonstrates that, for a 24-hour phototherapy, a decrease in the transcutaneous bilirubin measured using a corresponding transcutaneous bilirubin meter can be detected.

The fact that devices are commercially available with which bilirubin can be measured, such as the Dräger JM-105 jaundice meter, should be mentioned.

Optical measurements of bilirubin in living tissue are, however, normally strongly influenced by the optical properties of the skin—for example, due to light absorption by hemoglobin, or pigments such as melanin—and these influencing factors can have different effects from patient to patient. In addition, differences can also occur in the same patient depending on blood flow at the moment, blood oxygen saturation, and the specific examined region of living tissue. It has thus been proposed to measure the thickness of an examined tissue region mechanically (cf. US 2013 00 23 742 A1). However, to some degree, this approach is significantly inadequate, especially since the tongs-like mechanism proposed there for the thickness measurement is not entirely practical for many applications—such as in newborns.

However, the previously known methods for bilirubin measurement do not only suffer from inaccuracies with respect to the measured value obtained due to skin pigmentation or perfusion. The optical properties themselves of the different forms of bilirubin can also be changed by different influencing factors, such as pH or a protein content of the solution. Effects, such as so-called Förster resonance, are relevant in this case, which cause the spectral properties of a molecule to shift according to the manner and concentration of other substances located in the vicinity. Thus, it was shown in vitro that the protein content of a bilirubin solution has an influence on the spectral properties. The fact that, in the case of transcutaneous measurement, variables such as the protein content are not known, generally makes it more difficult to determine bilirubin, and makes it very problematic to distinguish between different forms of bilirubin.

In view of these difficulties, it is not surprising that common methods are hardly able to readily distinguish direct from indirect bilirubin, particularly if it is required at the same time that a corresponding determination must be possible quickly, and optionally without the attendance of specifically trained personnel, such as physicians, nurses and the like, using an inexpensive device which may be used continuously over a longer period of time.

It would accordingly be desirable to make possible a simple determination of bilirubin with which at least some of the existing problems can be at least partially solved.

### SUMMARY OF THE INVENTION

One aspect of the present invention relates to providing a novel solution for commercial application.

Accordingly, embodiments of a method and a device for the transcutaneous measurement of bilirubin are disclosed herein.

According to a first basic idea of the invention, it is thus proposed that, in a method for determining bilirubin, in which light is irradiated into a living tissue region with a wavelength which is suitable for the transformation of bilirubin, light from the irradiated living tissue region is detected and a time sequence of detection signals changing according to the transformation is determined, and bilirubin is determined in response to the time sequence, further provision is made so that light is irradiated locally into the living tissue region, the intensity of which is high enough to locally degrade, in comparison with other tissue regions, a more rapidly phototransformable form of bilirubin compared to a more slowly phototransformable form of bilirubin, regardless of the body's own transport processes, in a manner which can be recognized with the detection means used, and provision is made so that different forms of bilirubin in the non-degraded state are quantified from the detection signals of the time sequence detected during the local degradation.

The irradiation can take place simply from the outside, i.e., transcutaneously. A tissue region can be irradiated, and its fluorescence can be determined outside the body. It should be mentioned that this accordingly purely optical method has advantages. For example, for the patient, unlike blood collection, it is pain-free, and a risk of infection is reduced since the skin barrier is not punctured. The reduction in the risk of infection is advantageous both for patients and for medical personnel, which would otherwise be exposed to an increased risk of infection when the blood samples are collected and handled.

The detection of the light obtained from the tissue region with one or more detectors is preferably carried out somewhat at a distance from the light source, so that a sufficiently long light path through the living tissue region is ensured, and a direct irradiation of light from the light source into the detector is prevented. Also to be noted is the fact that an absolute intensity can optionally be detected directly with an additional sensor, for example for standardization purposes. Insofar as fluorescence signals are to be detected, it should be noted that continuous illumination is nonetheless desirable during the capture of the time sequence in order to achieve the continuous phototransformation, in particular of the more-rapidly phototransformable bilirubin. For this purpose, sufficient light must be irradiated throughout the entire measurement—i.e., over the entire time sequence. With sufficient fluorescence, it would be possible to use a pulsed light source for this purpose, which, during the measurement and in particular during or before each detection of an individual detection signal of the time sequence, radiates at least one or more (and intentionally relatively strong in each case) light pulses toward the tissue. However, a structurally simpler, and therefore preferred approach is a continuous operation of the light source during the capture of a measurement sequence.

It should be mentioned that the light irradiated into a living tissue region for the measurement is locally irradiated, i.e., over a small surface area relative to the total skin surface of the patient. The phototherapy for bilirubin degradation, however, uses light irradiated over as large a surface area as possible. Typically, the surface area of the local irradiation is in the present case less than 5 cm², preferably less than 3 cm², in particular about or less than 1 cm². This also allows the use of small and thus continuously portable devices with which the method is realized.

A small irradiation surface area of below 5 cm², preferably below 3 cm², preferably about 1 cm² allows comparatively high irradiation power densities with low structural complexity—which in turn allow the desired rapid bilirubin transformation. In one practical embodiment, the illumination used (blue) LEDs, which were operated at a power of from 75 mW to 150 mW. However, it should be mentioned that, in the case of an excessively small irradiation surface, small-area pigment spots on the skin have too much of an influence, and that irradiated (capillary) blood vessels in the beam path between light source and light detector have a potentially stronger effect, which is undesirable.

As regards the speed of the bilirubin transformation, this transformation takes place quickly in such a way that, due to the body's own transport processes, such as diffusion and capillary transport, the bodily fluid, such as blood, which has not been irradiated is not transported to a significant extent to the location of the irradiated living tissue, which would prevent a local degradation of the phototransformable forms of bilirubin proceeding at different rates, as detectable in the time sequence. It is clear that the degradation takes place more quickly at relatively high light intensities, and that the body's own transport processes for different patients or patient groups also occur at different speeds. The absolute minimum light intensity required for the measurement is thus also higher for certain patients or patient groups than for others. The upper limit is determined by the light intensity that can be scattered where damage is caused by the radiation—or, where the degradation takes place so quickly that it is no longer resolvable with a given sensor system, or where there are limits for generating the light to be irradiated.

It should also be mentioned that in contrast to the present method, it is generally desired in phototherapy that a body's own transport process takes place during the irradiation, since this contributes to an efficient bilirubin degradation that is in fact therapeutically desired in phototherapy. The method of the present invention should therefore not be confused with repeated measurement of bilirubin levels during long-term, phototherapeutic irradiation, and particularly not if bilirubin measurements are repeatedly carried out in parallel with such a phototherapeutic irradiation-optionally also by local irradiation of light, as may be known from the prior art. The time sequences detected in the known phototherapy in fact detect at most a gradual drop in the total bilirubin overall value, but without determining the different forms of bilirubin from a measurement-radiation-induced local drop in more rapidly transformable forms of bilirubin, or distinguishing these different forms of bilirubin from one another.

As regards the quantification of the different forms of bilirubin, it should be noted (cf. FIG. 5, taken from the prior art) that the spectra of conjugated and unconjugated bilirubin are very similar, especially if fluorescence spectra of bilirubin in fluid are absorbed only with a spectral resolution that can still be achieved in daily medical practice without increased outlay. A direct spectral distinction is thus difficult. The invention has recognized and taken into account that the different photodecomposition in light of conjugated and unconjugated forms of bilirubin is utilized for quantification of different forms of bilirubin, especially since it is possible to use blue light of a wavelength at which substantially unconjugated, indirect bilirubin is degraded, at which conjugated bilirubin is degraded at least less rapidly, and at which other molecules which otherwise will interfere with spectroscopic analyses, such as melanin or hemoglobin, are not significantly destroyed—at least when using reasonably limited intensity. It should also be mentioned that the faster degradation of indirect bilirubin, such as Z,Z isomers, by the effect of light from the blue to green region is used therapeutically, since the isomers produced by the effect of light have a water solubility which is increased compared to indirect bilirubin, so that the bilirubin can also be excreted in the liver without prior metabolism—which leads to achieving the therapeutically desired bilirubin reduction. In this respect, lumirubin and the Z, E, and IXα bilirubin isomers may be mentioned as the most important degradation products.

Due to the comparable spectral properties of conjugated and unconjugated bilirubin (cf. FIG. 5), the fluorescence spectra are also comparable. However, the degradation of bilirubin leads to a decrease in fluorescence during the time sequence, because a clearly noticeable degradation of bilirubin takes place due to the lack of transport of unirradiated body fluid to the location of the local irradiation. In a time sequence captured with sufficient time resolution, the degradation of the rapidly degradable bilirubin thus results in a rapid decrease in the fluorescence. Accordingly, it is possible to deduce the presence of more or less unconjugated, indirect bilirubin from the decrease behavior of the fluorescence. It should be noted that the fluorescence does not completely drop to zero; rather, a sufficiently strong drop can nevertheless be achieved at reasonable irradiation intensities and radiation power densities.

The quantification does not necessarily have to take place absolutely, and therefore does not necessarily have to supply an absolute value in mg/dl. It is often already helpful to be able to indicate a particularly high or particularly low proportion of unconjugated bilirubin of the total bilirubin. A total bilirubin value can thereby be determined from the total fluorescence itself, for example at the beginning of the measurement, or the average total fluorescence over the course of a certain measurement period can be determined. The specification “unconjugated bilirubin high” or “unconjugated bilirubin low”, and/or, for example, an indication of “unconjugated bilirubin lower than in the normal range”, “unconjugated bilirubin higher than in the normal range”, or “unconjugated bilirubin in the normal range”, is thus already understood as a quantification, since this rough information can significantly facilitate a diagnosis. However, it should be emphasized that, as a rule, a quantitative determination of total bilirubin, indirect bilirubin and direct bilirubin is possible, namely with an accuracy that makes it possible to differentiate between different causes of the hyperbilirubinemia, such as liver/biliary tract problems or intestinal problems. It should also be mentioned that, in various passages of the present disclosure, reference is made by way of example to (newborn) jaundice as contextualization for why the measurement of bilirubin and, if appropriate, the distinction of different forms of bilirubin, is useful for diagnostic purposes. However, it should be noted that the proposed and disclosed measurement of bilirubin, and the distinction of different forms of bilirubin, themselves involve a diagnostic benefit before bilirubin values are elevated enough to produce a clearly recognizable coloring of the skin. In this respect, it should be emphasized that there is a diagnostic benefit in the method disclosed here and in the devices disclosed here, both in terms of measurements of the total bilirubin on the basis of measurements which are carried out at a plurality of different wavelengths, and also in terms of measurements of different forms of bilirubin based on time sequences, even at rather low bilirubin values, i.e., this is in no way a purely niche application. Rather, in many cases, classic laboratory methods used for analyzing blood samples can be dispensed with, thanks to the methods and devices disclosed here. In other words, the accuracy and sensitivity of the method and device according to the present disclosure are high.

It will be apparent to a person skilled in the art that quantification of the different forms of bilirubin based on the time sequences requires that the corresponding detection signals captured at the points of the time sequence be considered more precisely, which is typically done by feeding the corresponding detection signals, possibly after signal conditioning, such as bandpass filtering, impedance matching, and analog-to-digital conversion, as an input signal, into a suitable evaluation algorithm or AI evaluation filters or the like, and that hardware suitable for such operations must be provided.

It should be noted that substances, such as lumirubin, are formed during the degradation of bilirubin. In addition, the configurational photoisomerization, and/or the photoxoidation, in which dipyrroles form, are of importance in the transformation of bilirubin with light irradiation. Some of the corresponding products can also contribute to the detection signals, and thus influence the time sequence. For this reason, it is often desirable to capture a time sequence at more than one detection wavelength.

It is preferred if the measurement duration for the capture of a time sequence is less than 15 min, preferably less than 10 min, in particular preferably less than 5 min, and very particularly preferably is less than 2 min. A measurement duration which is too long will result in the body's own transport processes which interfere with the actual measurement gaining in importance—in the case of typically-irradiated living tissue regions, which may lie at the forehead, a finger, or the arm, and which are generally quite well perfused by diffusion processes and the like. It can be seen that said body's own transport processes have a stronger effect in tissue regions that are particularly well perfused. These effects have a less pronounced effect on a shorter measurement duration. In addition, it is generally desirable for patients and possibly also for medical personnel to not have to wait for a result too long. Therefore, measuring times of less than 2 minutes, for example of 1 minute, are particularly preferred. Within this measurement duration, however, a time sequence with an adequate number of detection signals has to be captured, which also should not be excessively impaired by noise. It is desirable that the evaluated time sequence comprises at least 5, preferably at least 10 detection signals. It should be mentioned that, depending on the blood flow of the irradiated tissue regions, the pulse may possibly have a disruptive effect because the pulse changes the optical properties of the tissue per se, even without bilirubin degradation. For this reason, sufficiently long measuring periods with an adequate number of detection signals are clearly preferred in a time sequence.

Accordingly, it is also preferred if the measurement duration for the capture of a time sequence is greater than 15 seconds, preferably is at least 30 seconds and in particular is preferably at least 50 seconds—better, at least 1 minute.

It is also preferred if the integration time per detection signal of the time sequence is at least 100 ms, preferably at least 500 s, in particular at least 1 second. In this regard, it should be mentioned that, regardless of the preferably rather high intensities and power densities of the irradiated light, a comparatively small quantity of fluorescent light can be detected, which leads to detection signals being comparatively highly noisy in the case of economically and technologically justifiable effort with respect to the detectors. A sufficient integration time per detection signal reduces this noise in a known manner, especially since it is also averaged over a pulse beat.

However, the integration time per detection signal should not become too large, because otherwise not enough measured values can be detected in a time sequence during the reasonable, available measurement duration—i.e., before transport processes noticeably and disruptively compromise a further measurable change in the detection signal, such as for example a fluorescence signal decrease due to a reduction of more rapidly phototransformable bilirubin. In a preferred embodiment, the method is therefore designed such that the time resolution of the time sequence is better than 10 seconds, preferably better than 5 seconds, in particular preferably better than 2 seconds, and in particular preferably approximately 1 second.

In a practical implementation, for example, a series of 30 fluorescence intensity measurements—in each case 1 second long—was detected over a measurement duration of 30 seconds, which produced good results.

It is possible and preferred if a repeated measurement of bilirubin is carried out, wherein, between the capture of two time sequences, a time of at least 5 minutes, better 10 minutes, preferably at least 15 minutes is waited, and during this waiting time the light intensity of the local light irradiation is reduced, wherein preferably the intensity of the light with which the more rapidly phototransformable form of bilirubin is locally degraded, in comparison with other tissue regions, is below 20% of the light intensity used for the measurement, preferably below 10% of the local light irradiation, and in particular preferably a light source locally radiating light radiates no light onto the living tissue region during the waiting time. Such a repeated measurement can be advantageous, in particular, in cases where a phototherapy is carried out in which the patient is irradiated with bilirubin-degrading radiation over a large area, and not only, as for the present measurement, locally, and in which the bilirubin degradation achieved therapeutically is to be detected. In such a case, regardless of the decrease in the bilirubin concentrations progressing in the entire patient with the (sufficiently intense) local irradiation during a first measurement, a local degradation of the more rapidly phototransformable bilirubin, i.e., of the unconjugated indirect bilirubin, is carried out, which leads to a characteristic change over the time of the detection signals of a first time sequence associated with the first measurement. Thereafter, the local, intense light source, which optionally has a locally particularly intense light in addition to the phototherapy carried out, can be switched off, whereupon, as a result of the body's own transport mechanisms, the distribution of different forms of bilirubin, as characteristic for the comparable, non-locally irradiated, regions, is gradually established again at the location of the irradiation. The speed of recovery of the forms of bilirubin at the location of the irradiation, reaching the globally-observed distribution, is dependent on the efficiency of the transport mechanisms, and can thus vary from patient to patient. The specified waiting times between 2 time sequences, of at least 5 minutes, better 10 minutes, and preferably at least 15 minutes, take this into account. As a rule, for newborns, it can be expected that after 15 minutes the recovery has occurred which is required to be able to determine a new time sequence. A second time sequence can then be captured for a second measurement. In this way, bilirubin measurements can thus be carried out repeatedly with the present method, wherein not only a measure of the bilirubin values gradually decreasing during the large-area irradiation of the patients during phototherapy is obtained, but also the different bilirubin forms/states can also be newly quantified in each case.

Due to the purely optical measurements, there are also no concerns about restrictions resulting from the limitation of the number of blood samples that can be reasonably taken in newborns and infants. It should be mentioned that, in human patients, at which the invention is primarily targeted, as in newborns and infants, but also in the case of small experimental animals, such as, for example, mice, problems of excessive blood collection can be avoided.

In addition, it should be noted that the measurement is strongly impaired by pigmentation of the skin, for example due to melanin present in the skin, since melanin does not only have similar spectral properties to those of bilirubin, thus disrupting the measurements, it also blocks the entry of light into the skin. That is to say that dark skin allows the passage of less blue light to the tissue, which impairs the bilirubin transformation significantly. Especially where multispectral measurements are carried out using suitable sensors, such base pigmentation of the skin can, however, be determined by illumination with light of one or more wavelengths which do not (or only very slightly) transform bilirubin, and by detection of the corresponding sensor signals. For this purpose, it is merely necessary for the light source to emit one or more colors in addition to blue used for the bilirubin transformation, which is readily possible with suitable multicolor LEDs. Detection signals can then be detected at the multispectral detectors if there is such an illumination, which allows a conclusion to be drawn about the skin pigmentation—and thus a correction of the bilirubin values to be determined with respect to the pigmentation. Such a correction measurement can take place directly before a first bilirubin measurement or subsequently thereto, or during a waiting time up to a subsequent measurement.

In a preferred variant, the light irradiated into the bilirubin transformation is a wavelength of 400 to 450 nm, in particular 400 nm. The wavelength and/or the wavelength distribution should be selected such that light can be generated on the one hand with a favorable and energy-saving light source, and, on the other hand, the light irradiated into the tissue can effect an efficient bilirubin transformation of one of the forms of bilirubin. This is the case for a wavelength around 400 nm because, on the one hand, light of this wavelength can be generated by means of LEDs with sufficiently high intensity and low energy consumption—also in a small device; and because, on the other hand, this wavelength is particularly efficient in order to achieve a rapid phototransformation of unconjugated bilirubin and thus a decrease in the returning fluorescence during the capture of a time sequence.

It is possible and preferred for light of a plurality of distinguishable wavelengths to be detected from the irradiated living tissue region, wherein preferably both light of the irradiation wavelength and, in different ways, longer wave light are detected. The detection of light of the irradiation wavelength initially allows an estimate of how much light is irradiated into the tissue and passes through it up to a detector. This is of importance because, for example, both the fastening of an arrangement on the patient's body and, for example, the pigmentation thereof can have a considerable influence on how much of the light generated by a light source is actually available for the phototransformation of bilirubin. This accordingly allows for a standardization of the fluorescence intensity.

It is also preferred that light is detected in at least two distinguishable wavelength ranges which do not comprise the irradiated light, and preferably comprise one of the wavelengths within an FWHM region which is selected from among the (central) wavelengths 500+−20 nm, 550+−10 nm, 570+−10 nm, 600+−20 nm and 650+−20 nm. In addition, light of the irradiation wavelength can be detected, for example, at 450+−10 nm, so that the detected intensities can be standardized. It should be mentioned that different time curves of the fluorescence profiles can result in different wavelength regions—for example, because the photolysis products generated during a phototransformation fluoresce to a different extent upon irradiation with the scattered light. It should be mentioned that a measurement in a plurality of wavelength ranges requires at most negligible additional complexity, because there are detector components which have a plurality of separate photodetectors, for example photodiodes, with respectively different upstream wavelength filters. An example is the AMS module AS7262, for receiving six different spectral channels, which has been used in a practical embodiment, wherein the wavelengths 500 nm, 550 nm, 570 nm, 600 nm and 650 nm were implemented as central wavelengths of the corresponding spectral channels with an accuracy of plus/minus 5 nm, and an FWHM bandwidth of 40 nm per channel. The evaluation of the detection signals detected on a plurality of these channels makes it possible to increase the accuracy of the bilirubin measurements. By considering a plurality of channels, inter alia, the absolute bilirubin concentrations can be detected more precisely than is possible when only one single channel is considered. Furthermore, it should be noted that irradiation of blue light as the radiation transforming bilirubin forms is not mandatory. Current phototherapy lamps, with which a bilirubin transformation is likewise achieved by radiation, primarily work at 450 to 470 nm. However, it is also worth considering whether light of longer wavelengths, 490 to 510 nm (i.e., light of the colors turquoise or green) could be better suited for the bilirubin degradation (cf. Hendrk J. Vreman et al., The effect of light wavelength on in vitro bilirubin photodegradation and photoisomer production in Pediatric Research (2019) 85:865-873). Although the present disclosure relates in some passages to a certain wavelength used for bilirubin transformation, such as 400 nm or 450 nm, this occurs primarily in order to avoid having to portray to the reader all potential possibilities of usable wavelengths at every point at which a short-wave wavelength used for the bilirubin transformation is referred to, and rather to be able to refer quickly to a short-wave wavelength that can be used for the bilirubin transformation. Therefore, a bilirubin transformation with light which is longer than 400 nm or 450 nm is not to be ruled out explicitly. It should be expected in that case that, where for example a wavelength of 510 nm is used as a wavelength for the bilirubin transformation, the fluorescence intensities will likewise be longer than specified above.

In a preferred embodiment, the method is designed such that light of the irradiation wavelength and longer wave light is detected, and the intensity of each detection signal of the detected longer wave light is related to the intensity of light of the irradiation wavelength detected, the time sequence thus obtained of the intensity of longer wave light and/or the intensity of the detected light of the irradiation wavelength, is used to deduce the non-degraded ratio of different forms of bilirubin. In other words, for each wavelength on which fluorescence is to be detected, the irradiated light intensity is first standardized before the different forms of bilirubin are quantified. This is useful insofar as the circumstance is taken into account that a stronger irradiation of transforming light into the tissue leads to a more rapid phototransformation, i.e., the relative decrease in fluorescence depends not only on the initial ratio of the different forms of bilirubin, but also on the intensity of the irradiated light.

It is possible and preferred for the respective intensities to be related to the intensity of the detected light of the irradiation wavelength for at least two different wavelengths which are longer than the irradiation wavelengths, and then for the non-degraded ratio of different forms of bilirubin to be deduced from the at least two irradiation-intensity-corrected time sequence values together. Such a procedure is useful insofar as, for each of the wavelengths, it is initially possible to infer separately the different forms of bilirubin, and then to calculate a variable taking into account the corresponding quantifications. It should be noted in any case that where a plurality of fluorescence wavelengths is to be evaluated in order to achieve quantification of the different forms of bilirubin, the detection signals are preferably standardized to the irradiated light intensity. As regards the standardization of the detection signals, it is also possible, in particular, to standardize to the amount of light irradiated in total upto a detection signal of a time sequence during a time sequence—i.e., the irradiation intensity integrated over time—and/or to take into account both the time integral of the irradiation intensity and the current irradiation intensity at the time point of the fluorescence, provided that the radiation intensity fluctuates greatly over the time of the time sequence.

It should also be mentioned that the change of the detection signals during a time sequence, revealed, for example, in the decrease in fluorescence, is not the only conclusive factor. Rather, the absolute strength of the fluorescence, or the absolute bilirubin concentration that can be deduced therefrom, can also be of diagnostic interest. It should be mentioned that it is thus possible to determine a value that is indicative of the total bilirubin. In this case as well, i.e., in the determination of a value for the total bilirubin integrative value, for example in the determination of an absolute total bilirubin concentration, it is advantageous to evaluate detection signals at a plurality of wavelengths together. It has been found that, when detection signals at a plurality of wavelengths are evaluated together, the correlation between the bilirubin measurements carried out optically by local transcutaneous irradiation of light into living tissue and the detection of fluorescent light from the living tissue is improved, and the bilirubin measurements carried out by laboratory analysis of blood sampled is improved.

It is particularly advantageous if ratios of the intensities obtained at different wavelengths are determined. For example, when irradiated with light of the wavelength 400 nm (blue), the ratio of the detection signals obtained for orange (600 nm) and red (650 nm) can be determined, and the ratio of the detection signals obtained for green (550 nm) and yellow (570 nm), or the ratio of the detection signals obtained for 450 nm and 550 nm, can be determined. In this way, a plurality of estimates of the total bilirubin can be obtained with one and the same time sequence due to the values detected at different locations of the spectrum. Such a procedure leads to values which are worse for a single color pair, such as blue/green or orange/red, than if extinction coefficient and layer thickness are determined precisely and the values are used under the assumption of a Lambert-Beer dependency for the bilirubin value determination. However, in practice, it is difficult to determine extinction coefficients of layer thicknesses precisely, which is why it is necessary to work with estimates—which, however, are influenced by the significant variations in, for example, skin pigmentation, different blood perfusion at the location of the measuring point, and greatly different skin structure from patient to patient, and therefore are usually very imprecise. The fact that newborns have a completely different skin structure to adults is only mentioned in a supplementary manner in this context.

In view of these inaccuracies given in principle, the proposed simultaneous consideration of individual total bilirubin values determined in different spectral pairings and the determination of a multispectrally determined total bilirubin value derived from the individual total bilirubin values, for example by mean formation, has an advantage in that the different pairings partially overestimate and partially underestimate the corresponding influencing variables, so that, given suitable averaging from the combination of the total bilirubin values obtained for a plurality of color pairs with a multispectral total bilirubin value, a variable can be determined which correlates very well with total bilirubin values obtained in blood analyses in the laboratory, even though each value which belongs to a single color pair is rather inaccurate in itself. In this respect, the additional effort for implementing a multispectral determination method for the total bilirubin is extremely low. As such, the total bilirubin value determined from the blue/yellow ratio is rather greater than the laboratory value determined analytically from blood, whereas the total bilirubin value determined from the color pairing green/yellow is rather lower than the laboratory value determined analytically from blood—yet the average value corresponds to the laboratory value determined analytically from blood of the total bilirubin.

Even more precisely, the total bilirubin value can be determined if at least the layer thicknesses are estimated from the detection signals, which becomes possible in the case of multispectral evaluation. Since ultimately only extinction coefficients and layer thickness are required as unknown variables in order to take into account the Lambert-Beer laws in the total bilirubin determination, it is in principle possible to determine these two variables from detection signals determined in a plurality of colors by suitable mathematical methods. Thus, the thickness of the layer, to which the irradiated light intensity is propagated, and which is accordingly irradiated by fluorescence light, is the same for all colors. This allows the path length to be determined using suitable mathematical methods, such as the Gaussian method. However, differences with respect to the extinction coefficient are quite likely to be expected for different wavelengths, but can either actually be ignored, regardless of the real variations from patient to patient, or taken into account in a standardized manner. As a result, a precise total bilirubin determination is made possible by the multispectral total bilirubin determination regardless of the large influence of the layer thickness and of the extinction coefficient for human tissue—which is only inaccurately determined for human tissue—which should be known when using Lambert-Beer's law. It should be mentioned that, after such an exact overall bilirubin determination, the absolute values of the direct and indirect bilirubin can also be determined. It should be mentioned that the presence of degradation products, such as lumirubin, would also be detectable with multispectral methods.

The fact that it is possible and preferred for direct and indirect bilirubin to be quantified as different forms of bilirubin results from the foregoing.

In addition, protection is also claimed for a device for measuring bilirubin, in particular according to a measurement method as described above, having a light source for irradiation of light into a living tissue region, with a wavelength suitable for the transformation of bilirubin, and with an intensity sufficiently high so that a more rapidly phototransformable form of bilirubin is locally degraded compared to a more slowly phototransformable form of bilirubin, in comparison with other tissue regions, regardless of the body's own transport processes; a detection arrangement for generating a time sequence of detection signals relating to the detection of light from the irradiated living tissue region, and an evaluation unit for evaluating a time sequence of detection signals in such a way that the non-degraded ratio of different forms of bilirubin can be deduced from the detection signals of the time sequence detected during the local degradation. The described construction of a claimed device shows that it is possible to provide particularly valuable information about different forms of bilirubin with little structural complexity. It can be seen from the illustration of the preferred embodiments of the method that neither the light source nor the detection arrangement has to be particularly complex. The signal conditioning of the detection signals is also simple and possible with little structural complexity. In a practical implementation, the detection signals are digitized, optionally after suitable bandpass filtering, impedance matching, and amplification. In view of the low time resolution of the time sequence, this digitization does not place any special requirements on an analog-to-digital converter; and also the subsequent evaluation of the data can be carried out on very inexpensive hardware, since only a few measured values have to be processed within long time periods.

With regard to optical components, it is preferred if, in the device for measuring bilirubin, the detection arrangement has a spectral filter means in order to be able to distinguish received light of different wavelengths from one another. However, it has already been pointed out purely by way of example that there are integrated components which can be used for hardware implementation, and even have a plurality of detector elements of different spectral sensitivity. The fact that lenses and possibly further optical elements in the beam path between the (LED) lighting means for generating the light to be irradiated and the skin, as well as in the beam path between the skin at the exit point and the detectors, are possibly required, should also be mentioned, along with the fact that these further required elements do not require excessive structural complexity.

In the case of a device for measuring bilirubin, it is further preferred to this extent, and also does not lead to a significantly more complex arrangement, if the irradiation location is spaced apart from the detection arrangement, wherein between the irradiation point and one—or each—light-sensitive detector surface, there is preferably a distance of at least 3 mm, preferably at least 4 mm, in particular at least 5 mm. It can be seen that such a spacing ensures that both the short-wave light irradiated for excitation and the longer wave light detected in response pass through a sufficiently long path of the tissue, so that strong signal detection signals can be detected. The light source should be aligned in such a way that it radiates away from a light source carrier, such as a printed circuit board as a carrier of a light source LED, and indeed steeply enough for the incoming light to penetrate sufficiently deeply into the tissue. At the same time, however, it should also radiate in the direction of the detectors, since more light is thus irradiated closer to the detectors in tissue regions. If this is taken into account, excessively large distances between detectors and light sources would lead to an excessively flat irradiation, which impairs the penetration of light into sufficiently deep tissue layers. The fact that the detector can optionally be mounted obliquely or can be provided with a suitable lens so that it receives more light from the irradiated region, is also mentioned.

Furthermore, this requirement also ensures that, within an arrangement, the detector arrangement can be sufficiently protected against scattered light propagating from the light source to the detector arrangement within the device. In a practical implementation, a distance between the detector arrangement and the light source of 0.5 cm has proven to be sufficient, allowing the use of SMD components to achieve a total size of a practical implementation of less than 3 cm.

Furthermore, in a device for measuring bilirubin, it is further preferred that a light source with discrete spectrum is used, in particular a semiconductor light source. The suitability of LEDs or semiconductor lasers is particularly mentioned.

In a device for measuring bilirubin, it is further preferred that at least the light source and the detection arrangement are arranged jointly on a carrier with which the device can be held in the region of the local living tissue, in particular directly on the skin of a patient. Typically, the light source and detection arrangement can be arranged on a printed circuit board as a carrier. For example, belts can be arranged on the carrier or on a housing surrounding it in order to wrap the arrangement around the patient in the manner of a cuff. Alternatively, the arrangement can also be applied to the skin with a larger piece of medical adhesive tape and/or bandaging. The arrangement is thus preferably directly in contact with the skin. The fact that this entails corresponding requirements for skin compatibility, and thus for the used carrier material and/or housing material, is apparent to a person skilled in the art.

It is also possible and preferred for the device claims to have a communication interface for wireless transmission of detected signals and/or of data generated in response thereto. In this regard, it should be mentioned that the arrangement can be designed not only in a structurally simple manner, but also that it has an extremely low energy requirement, accordingly allowing measurements with commercially available small batteries over a longer period of time. Thus, by means of the wireless transmission of detected signals or data generated therefrom, a considerable additional convenience compared to wired solutions is made possible-especially for measurements of longer duration.

### BRIEF DESCRIPTION OF THE DRAWINGS

The invention is described below only by way of example, with reference to the drawings, wherein:

FIG. 1 is a device for bilirubin measurement according to the present invention;

FIG. 2 is a fluorescence curve captured with a device according to FIG. 1, in which a decrease of the detected fluorescence intensity is apparent over time;

FIG. 3 is a fluorescence profile for light from the wavelength range around 570 nm, captured with a device according to FIG. 1;

FIG. 4 is a comparison of total bilirubin values which were determined from the ratio of the light intensities captured at 450 nm and 550 nm, with total bilirubin values which were determined with blood analysis in the laboratory.

FIG. 5 is fluorescence spectra in solution of conjugated and unconjugated bilirubin according to the prior art.

### DETAILED DESCRIPTION

FIG. 1 shows a device 1 with which a method for determining bilirubin can be carried out in a simple manner, wherein light is irradiated into a living tissue region with a wavelength which is suitable for the transformation of bilirubin, light from the irradiated living tissue region is detected and a time sequence is determined according to the transformation of changing detection signals, and bilirubin is determined in response to the time sequence, and wherein light is irradiated locally into the living tissue region, the intensity of which is high enough to locally degrade, in comparison with other tissue regions, a more rapidly phototransformable form of bilirubin compared to a more slowly phototransformable form of bilirubin, regardless of the body's own transport processes, and provision is made so that different forms of bilirubin in the non-degraded state are quantified from the detection signals of the time sequence detected during the degradation.

In this case, the device 1 shown in FIG. 1 and generally denoted by 1 for measuring bilirubin has a light source 101 for irradiating light into a living tissue region vG, wherein the light irradiated into the living tissue region vG has a wavelength lambda 1 which is suitable for the transformation of bilirubin, and which further has an intensity which is high enough so that a more rapidly phototransformable form of bilirubin with respect to a more slowly phototransformable form of bilirubin, is degraded locally as compared to other tissue regions, regardless of the body's own transport processes. The device 1 further comprises, for generating a time sequence of detection signals which are related to the detection of light from the irradiated living tissue region, a detector arrangement 103, wherein the detector arrangement comprises a plurality of detectors 103a, 103b—which are separate in the present case—for light of different wavelengths, in the present case shown by the arrows Lambda 1 and Lambda 2, and an evaluation unit (not shown) for evaluating a time sequence of the detection signals in such a way that the non-degraded ratio of different forms of bilirubin can be deduced from the detection signals of the time sequence detected during the local degradation.

The light source and the detectors are arranged at a distance from one another on a printed circuit board 105 serving as a carrier, which also comprises the evaluation unit and an associated power supply such as a replaceable battery, and circuits associated with the detectors, with which electrical light detection signals generated by the detectors are amplified, impedance adjusted and digitized, so that they can be digitally processed as digital data by the evaluation unit. In the exemplary embodiment shown, the distance between the light source and the detector is 5 mm, which on the one hand allows sufficient shielding against light entering directly from the light source to the detector, and on the other hand enables an orientation of the light source and detector in such a way that light can penetrate into the living tissue sufficiently far and deeply. In this case, the light source radiates light obliquely into the living tissue, via its installation and/or the associated lenses, namely with an inclination toward the detector. The light source and the detectors are arranged such that their input and output lenses can be pressed directly against the living tissue. The irradiation surface area is less than 1 cm².

The carrier can be embedded in a plastic compound or provided with a housing such that it can be fixed at a selected location, for example by rubber bands or hook-and-loop fasteners. A fingertip is shown as the region of the living tissue, but this is not mandatory. Rather, other areas of the skin can also be selected, wherein, for example, regions near the wrist (where wristwatches are typically worn) are advantageous for long-term monitoring; other regions can offer advantages if the body's own transport processes run more slowly there due to a somewhat weaker blood circulation, and thus a fluorescence curve drop can be tracked over a longer period of time.

In the present embodiment, the light source has a wavelength of 450 nm, i.e., a wavelength for which it is well known that an efficient transformation, in particular of unconjugated bilirubin, can be achieved, and which can be produced easily with LEDs available on the filing date. However, reference is explicitly made to the usability of other wavelength ranges for bilirubin transformation.

In the present case, the detector is an integrated multispectral detector component with separate sensors for the central wavelengths 450 nm, 500 nm, 550 nm, 570 nm, 600 nm 650 nm. The different spectral sensitivity of the respective sensors is thereby achieved by upstream optical bandpass filters. It should be noted that modern photo sensors with Bayer filters or the like have pixels with different spectral sensitivity, and, assuming a sufficiently high sensitivity, could optionally be used, so that it is conceivable in principle to use cameras of smartphones as detectors, especially if they are close enough to a suitable light source.

The evaluation unit is designed not only to process the digitized sensor signals in such a way that the detection signals of all separate sensors, initially integrated over equidistant periods of 1 sec. in the present case—as preferred—are detected and stored, each over 1 second, until a time sequence of 60 values per spectral channel has been detected, and also to control the light source in such a way that the light source emits light continuously during the capture of the time sequence, and the light emission is likewise ended after the time sequence has ended. The evaluation unit can further be designed to capture a further time sequence after a certain time period, such as 15 minutes. It should be noted that the above-mentioned equidistant periods of 1 second, the integration time of 1 second, the number of 60 values per spectral channel in a time sequence, and the specified waiting time until the capture of a new time sequence are not absolutely necessary; if appropriate, other values, in particular adjustable values, can also be selected. In a preferred variant, a wireless interface, for example for communication via Bluetooth, is present in order, among other things, to carry out such adjustments and to transmit measured values. During the measured value transmission, the detection signals can preferably be locally evaluated, and then only a current total bilirubin measured value determined therefrom and the distribution of unconjugated bilirubin are transmitted. This is preferred in clinical practice. Alternatively, the individual detection signals can also be transmitted to check the device, for example by a service technician. It should be noted that, in addition to or instead of a wireless interface, a wired interface and/or a display can be provided on which information, such as a current total bilirubin measured value or the distribution of unconjugated bilirubin, can be displayed.

The arrangement is used as follows:

First, the device is attached in direct contact with the skin of a human patient, and then the light source is activated, so that light is irradiated locally into the living tissue in the skin. This light has an intensity and wavelength suitable for transforming a significant amount of bilirubin located there. In such a bilirubin transformation, light-sensitive forms of bilirubin, in particular unconjugated bilirubin, are degraded more quickly than other forms, such as conjugated bilirubin. This leads, on the one hand, to a change in the initial ratio of conjugated bilirubin to unconjugated bilirubin, on the one hand, and, on the other hand, since the bilirubin transformation is also associated with a degradation of the forms of bilirubin toward other compounds, such as lumirubin, this also leads to a decrease in the total bilirubin value within the small local region—in any case, as long as these changes are not compensated for in a measurable manner by the body's own transport processes via which the fluid in the irradiated living tissue region is collected. Such an exchange takes place, inter alia, by blood circulation, so that the total bilirubin degradation occurring in the blood, and brought about by the local irradiation and the changes in the ratio of different forms of bilirubin, can only be detected at comparatively very high intensities—whereas the collection takes place more slowly within the tissue. Nevertheless, the bilirubin present in the blood and its selective degradation, since living tissue is regularly perfused, contributes to the measurement signal, so that a measurement in blood can also be regarded as being covered, above all when sufficiently high intensities are used, by reference to a measurement of living tissue regions.

A measurement sequence of digitized detection signals is then detected during a one-minute measurement period, while the light source illuminates continuously with constant (target) intensity. With this series of measurements, on the one hand, for standardization to the intensity of the light of the irradiated light wavelength, light of the irradiation wavelength is detected and, on the other hand, light also of all other wavelengths is detected by the multispectral detector, wherein each measurement is integrated over a measurement duration of 1 second.

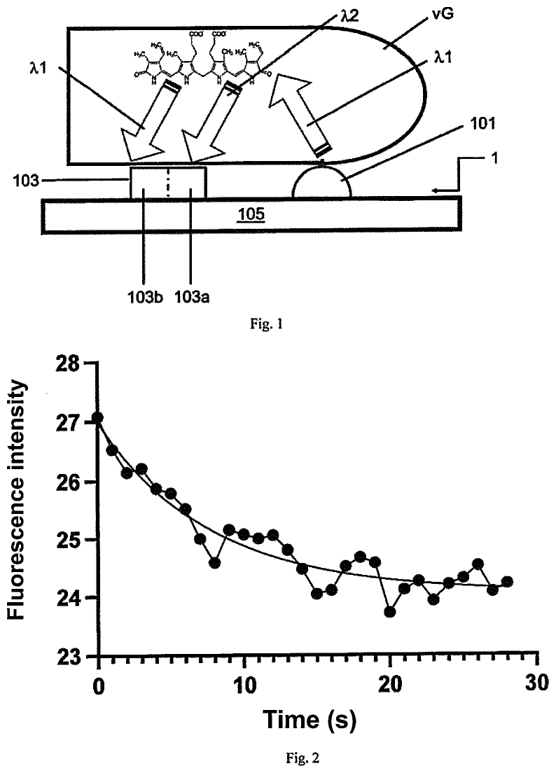

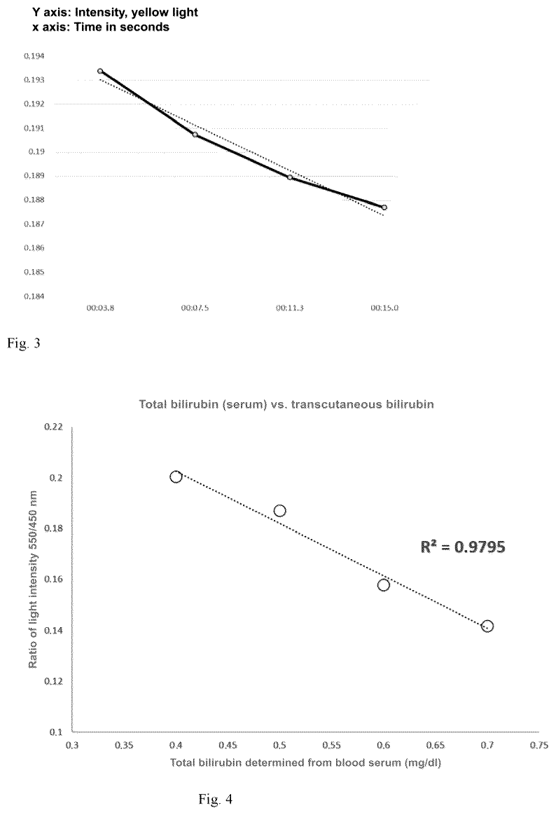

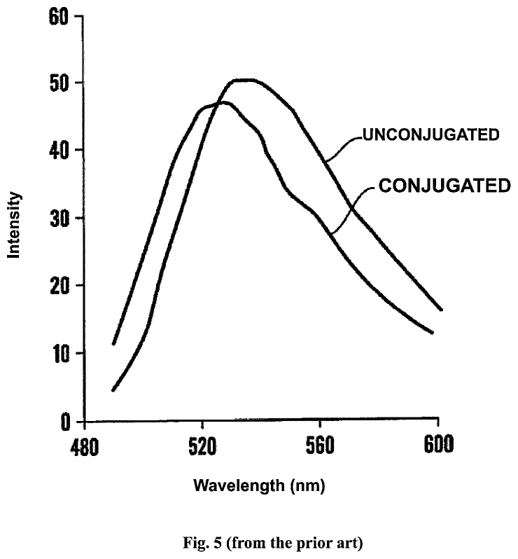

Time sequences are obtained as shown in FIG. 3 for yellow light, or as shown in FIG. 2 for the total intensity over all fluorescence channels for a somewhat longer period of the time sequence. FIGS. 2 and 3 clearly show that the fluorescence decreases during the capture of the time sequence, which can be attributed to the degradation of the forms of bilirubin by phototransformation. It should be noted here that the values shown in FIG. 2 are subject to periodic fluctuations, which can be attributed, among other things, to the pulse rate of the patient and the associated changes at the location of the measurement. Nevertheless, from the measured values of the time sequence, both the total bilirubin value and the ratio of different forms of bilirubin, i.e., in the present case of conjugated and unconjugated bilirubin, can be deduced with high accuracy.

After the capture of the time sequence, it is evaluated. For this purpose, the individual measured values are first standardized to the irradiation intensity. In principle, it is possible to determine the total bilirubin values from an individual fluorescence wavelength which is longer than the short-wave light irradiated for the bilirubin transformation; however—and it should be emphasized that this is considered to be inventive in its own right and, optionally, can be claimed separately in divisional applications—the ratios of different spectral pairs of the measured values obtained at the beginning of the measurement are preferably formed in order to determine a total bilirubin value. It is expressly disclosed that this type of total bilirubin value determination is also advantageous, even if not absolutely required, in connection with the distinction of different forms of bilirubin by considering the time sequence, in particular the decrease in fluorescence. In the case of the spectral pair formation, not only does the first measured value still influenced by the bilirubin transformation need to be optionally taken into account, but also, to reduce pulse rate effects and the like, some initial values can be considered together. Although the quotient formation of the (standardized) intensities detected at different points in the spectrum does not take into account the extinction coefficient and the layer thickness, a quotient can be calculated for multiple pairs of divergent wavelengths, and an associated total bilirubin value can be determined for this quotient. This determination of a total bilirubin value for a corresponding quotient can be compared to a previous calibration by laboratory measurements. A corresponding comparison curve is shown, for example, in FIG. 4 for the ratio of the detection signals at 550 nm/450 nm, wherein a measurement was used there in which the irradiated light was 450 nm—i.e., a standardization of the detection signals detected at 550 nm was substantially carried out to the irradiation intensity.

Via the total bilirubin measured values thus obtained, which result for the different spectral pairs, and which partially overestimate and partially underestimate the laboratory value for blood samples, an averaging can then be carried out. In the simplest case, this can be unweighted—but still offers high accuracy.

It should be mentioned that other methods of evaluation and determination of a total bilirubin value are also mentioned. Since ultimately the series of measurements for all wavelengths—at least as far as the irradiated light is concerned—are influenced by the same parameters of extinction coefficient and layer thickness, and the measurement series of all wavelengths are captured for the same total bilirubin value, a system of equations with a plurality of unknowns can optionally be set up, and the extinction coefficient or layer thickness can be determined therefrom, which in turn allows an even more accurate determination of the bilirubin value.

It is then possible to deduce the proportion of unconjugated bilirubin from the irradiated light intensity and the speed of the fluorescence decrease which is detected in the time sequence. The fact that the detected detection signals are also influenced by the degradation products, and that this can possibly lead to measurable effects that improve the measured values, should be mentioned.

After the total bilirubin value and the ratio of conjugated to unconjugated bilirubin are determined, these values can be displayed during the wait for a new series of measurements to be captured.

In summary, particularly valuable measured values are thus provided in a simple manner, and with little structural complexity.

Finally, it should be mentioned that the applicant reserves the right, optionally in divisional applications, to also claim protection for a method for the in vivo transcutaneous measurement of the content of bilirubin in body fluids, comprising the steps of irradiating light of a first wavelength lambda1 and known intensity from a light source (101) toward a living tissue region, detecting the intensity of the light emitted by bilirubin in the body fluids with a second wavelength lambda2 using a first sensor in a detector, a) detecting the intensity of the light of the first wavelength lambda1 returned by the living tissue region using a second sensor in the detector, b) portraying the ratio of the detected intensities of the light of the second wavelength lambda2 to the light of the first wavelength lambda1, and thereby eliminating non-relevant influencing factors, and c) calculating the content of bilirubin. It should be pointed out that transcutaneous measurement refers to a non-invasive measurement which is carried out through the skin without damaging the skin in the process, and body fluids in which the bilirubin is present can particularly be understood as blood, in particular the blood plasma fraction. However, it should be mentioned that bilirubin also occurs in tissue fluids. It should be noted that a reference to other tissue fluids is relevant when the phototransformation is considered, because in these the exchange takes place more slowly due to the body's own transport processes than is the case for blood, which is why a decrease in certain forms of bilirubin can be observed in tissue fluids. A living tissue region can be a part of the human body which in particular has skin which is normally perfused, as well as layers lying close to it—although this definition is also not mandatory. However, it should be mentioned in this regard that there is a series of factors only known in part, but sometimes also unknown, which influence the measurement, wherein, for example, the density of the living tissue region (vG) and layer thickness of the living tissue region (vG) belong to these factors. These factors are, on the one hand, dependent on the body location of the measurement, and on the other hand are very individual to the patient. It should be mentioned that this reservation of the applicant to claim for certain embodiments, if any, must not be interpreted as meaning that features which, with respect to these reserved methods and apparatus also assumed to be protectable, must also be present, let alone necessarily present, in the variants of apparatus and methods described above and claimed below.

In a preferred variant of the above method, it can also be provided that the irradiation of light with the first wavelength 21 in steps a) is carried out continuously in order to excite free bilirubin in the body fluids into a phototransformation, with conversion products of bilirubin, steps b) to d) are repeated at the same living tissue region in predetermined time intervals, step e) comprises the sub steps e1) of calculating the 1st content of bilirubin up to en) of calculating the nth content of bilirubin, further comprising the step f) of detecting the change in intensity of the light emitted by the bilirubin in the body fluids, of a second wavelength lambda2 using the 1st to nth contents of bilirubin, and correlating this with the content of indirect bilirubin.

It should also be mentioned that, in particular, embodiments must be claimed, according to which, in step b), the second wavelength lambda2 is used in the fluorescence spectrum of bilirubin in a second wavelength range between 500 nm and 600 nm in the region of yellow light, in particular at 550 nm. It should further be mentioned that the first wavelength lambda1 is in the region of blue light, in particular at 450 nm. It should also be mentioned that, in a preferred variant of the above method for which the applicant also claims protection, the light source has a discrete spectrum.

It should also be noted that the applicant also reserves the right to claim protection for a device for transcutaneous measurement of the content of bilirubin in bodily fluids, comprising a light source which is designed to generate light having a first wavelength lambda1 in the wavelength range between 400 nm and 500 nm with known intensity and to radiate it toward a living tissue region,—a detector which is designed to detect light having a second wavelength lambda2 in the wavelength range between 500 nm and 600 nm with a first sensor and light of the first wavelength lambda1 with a second sensor, separately from one another, wherein the light is returned in each case from the living tissue region,—a mounting element in which the light source and the detector are arranged at a predetermined angle and a predetermined distance from one another and to the living tissue region, and a computer unit. In such a device, it can then be advantageous that it has dimensions of 1 cm to 4 cm. In addition, it is possible to design such a device in such a way that it is integrated into a personal electronic device which a person wears directly on the skin.

The fact that such a device then is a communication interface for wireless transmission of data captured and generated in the computing unit is also mentioned, as is the usability of the device in telemedicine.

1-18. (canceled)

19. A method for determining bilirubin of human patients, the method comprising:
irradiating light of a wavelength suitable for transformation of bilirubin into a living tissue region,
detecting light from the irradiated living tissue region,
determining a time sequence of detection signals changing according to the transformation, and
determining bilirubin in response to the time sequence,
wherein light at a power from 75 mW to 150 mW is irradiated locally into the living tissue region, so that the intensity of which is high enough to locally degrade a more rapidly phototransformable form of bilirubin, also called unconjugated, indirect bilirubin, compared to a more slowly phototransformable form of bilirubin, also called conjugated, direct bilirubin, relative to other tissue regions, regardless of the body's own transport processes, by using blue light of a wavelength of 400 to 450 nm or longer, up to 510 nm, at which substantially unconjugated, indirect bilirubin is degraded, and at which conjugated bilirubin is degraded at least less rapidly, and
wherein different forms of bilirubin in the non-degraded state are quantified from the detection signals of the time sequence detected during the degradation by taking into account that the different photodecomposition in light of conjugated and unconjugated forms of bilirubin is utilized for quantification of different forms of bilirubin and whereas the measurement duration for the capture of a time sequence is less than 2 min.

20. The method according to claim 19, wherein a total bilirubin value is determined from the average total fluorescence over the course of a certain measurement period at the beginning of the measurement.

21. The method according to claim 19, wherein the measurement duration for the capture of a time sequence is greater than 15 seconds.

22. The method according to claim 19, wherein an integration time per detection signal of the time sequence is at least 100 ms.

23. The method according to claim 19, wherein the time resolution of the time sequence is less than 10 seconds.

24. The method according to claim 19, wherein a repeated measurement of bilirubin is carried out, wherein, between the capture of two time sequences, there is a wait of at least 5 minutes, and during the wait the light intensity of the local light irradiation is reduced.

25. The method according to claim 24, wherein the intensity of the light with which the more rapidly phototransformable form of bilirubin is locally degraded relative to other tissue regions is less than 20% of the light intensity used for the measurement.

26. The method according to claim 25, wherein a light source locally irradiating light irradiates no light onto the living tissue region during the wait.

27. The method according to claim 19, wherein light of a plurality of distinguishable wavelengths is detected from the irradiated living tissue region.

28. The method according to claim 27, wherein both light of the irradiation wavelength is detected and longer wave light distinguishable therefrom is detected.

29. The method according to claim 19, wherein light is detected in at least two distinguishable wavelength ranges which do not comprise the irradiated light, and, within an FWHM region.

30. The method according to claim 19, wherein light of the irradiation wavelength and longer wave light is detected, the intensity of each detection signal of the detected longer wave light is compared to the intensity of detected light of the irradiation wavelength, and, from the time sequence of the standardized detection signals thus obtained compared to the intensity of the longer wave light and to the intensity of the detected light of the irradiation wavelength, the non-degraded ratio of different forms of bilirubin is deduced.

31. The method according to claim 19, wherein, after each intensity for at least two different wavelengths which are longer than the irradiation wavelength has been compared to the intensity of the detected light of the irradiation wavelength, the non-degraded ratio of different forms of bilirubin is deduced from the at least two time sequence values corrected for the irradiation wavelength.

32. A device for measuring bilirubin of human patients with the method according to claim 19, the device comprising:
a light source for the irradiation of light into a living tissue region, with a wavelength of 400 to 450 nm or longer, up to 510 nm which is suitable for the transformation of bilirubin, and which has an intensity which is high enough so that a more rapidly phototransformable form of bilirubin is locally degraded, compared to a more slowly phototransformable form of bilirubin, relative to other tissue regions, regardless of the body's own transport processes;
a detection arrangement for generating a time sequence of detection signals which are based on the detection of light from the irradiated living tissue region; and
an evaluation unit for evaluating a time sequence of the detection signals in such a way that the non-degraded ratio of different forms of bilirubin can be deduced from the detection signals of the time sequence detected during the local degradation.

33. The device for measuring bilirubin according to claim 32, wherein the detection arrangement has a spectral filter in order to be able to distinguish received light of different wavelengths from one another.

34. The device for measuring bilirubin according to claim 32, wherein the irradiation location is spaced apart from the detection arrangement, wherein there is a distance of at least 3 mm between the irradiation point and one or each light-sensitive detector surface.

35. The device for measuring bilirubin according to claim 32, wherein the light source has a discrete spectrum.

36. The device for measuring bilirubin according to claim 32, wherein at least the light source and the detection arrangement are arranged together on a carrier on which the light source and the detection arrangement can be held in the region of the local living tissue.

37. The device for measuring bilirubin according to claim 36, wherein the light source and the detection arrangement can be held directly on the skin of a patient

38. The device according to claim 32, wherein the device has a communication interface for wireless transmission of detected signals and/or data generated in response thereto.
